(19) United States
(12) Patent Application Publication
(10) Pub. No.: US 2024/0228647 A1
(43) Pub. Date: Jul. 11, 2024

(54) OFATUMUMAB FOR TREATING MULTIPLE SCLEROSIS IN ASIAN PATIENTS

(71) Applicant: Novartis AG, Basel (CH)

(72) Inventor: Ratnakar Pingili, Southlake, TX (US)

(21) Appl. No.: 18/286,030

(22) PCT Filed: Apr. 13, 2022
(86) PCT No.: PCT/EP2022/059900

§ 371 Date: Oct. 6, 2023

Related U.S. Application Data
(60) Provisional application No. 63/174,765, filed on Apr. 14, 2021.

(51) Int. Cl. C07K 16/28; A61K 39/00; A61K 45/06; A61P 25/28

(52) U.S. Cl. CPC C07K 16/2887; A61K 45/06; A61P 25/28; A61K 2039/505; A61K 2039/545

(57) ABSTRACT

The invention concerns ofatumumab for use in the treatment of multiple sclerosis (MS), wherein patients are treated who are of Asian race. The invention further relates to ofatumumab for use in the treatment of multiple sclerosis, wherein the treatment is ethnically insensitive. The invention further relates to ofatumumab for use in the treatment of multiple sclerosis, wherein patients having certain genetic or physiological risk factors are treated.

The invention concerns ofatumumab for use in the treatment of multiple sclerosis (MS), wherein patients are treated who are of Asian race. The invention further relates to ofatumumab for use in the treatment of multiple sclerosis, wherein the treatment is ethnically insensitive.

The invention further relates to ofatumumab for use in the treatment of multiple sclerosis, wherein patients having certain genetic or physiological risk factors are treated.

Multiple Sclerosis (MS) is a chronic, immune-mediated disease of the central nervous system characterized by inflammation, demyelination and axonal/neuronal destruction, ultimately leading to severe disability. Although there is no cure for the disease, a variety of disease-modifying therapies (DMT) are available which usually slow down disease progression. The precise aetiology of MS is not yet known, although epidemiological data indicate that both genetic and environmental factors are important. The development of MS must start in individuals who are genetically susceptible. The importance of genetic factors in susceptibility to MS has been shown by genetic epidemiological studies.

Although most disease-modifying therapies for MS have traditionally been conceptualized as functioning via T cell-based mechanisms, a growing body of data indicates that these DMTs have demonstrable effects on B cells as well. Common themes include promoting naïve B cells rather than memory or plasmablasts (alemtuzumab); shifting B-cell cytokines towards an anti-inflammatory tone (beta interferon, glatiramer acetate, fingolimod); increasing B-regs (beta interferon, glatiramer acetate, fingolimod and dimethyl fumarate); decreasing class II MHC expression and costimulatory molecules on B cells required for antigen presentation (beta interferon and dimethyl fumarate); sequestering B cells in lymphoid organs (fingolimod); blocking VLA-4 mediated B cell trafficking to the CNS (natalizumab); or direct cytolysis of B cells (alemtuzumab, teriflunomide, mitoxantrone), see Greenfield et al., Ann Neurol. 2018 January; 83(1): 13-26.

Greenfield further reports that the monoclonal antibodies (mAbs) rituximab, ocrelizumab and ofatumumab which are each anti-CD20 antibodies are currently in clinical use for MS.

Rituximab, a chimeric mouse-human monoclonal antibody, was approved in 1997 for B cell lymphoma and indeed was one of the first mAbs ever developed for clinical use. Rituximab works primarily by depleting B cells through complement-dependent cytotoxicity (CDC), but also has significant antibody-dependent cellular cytotoxicity (ADCC) activity.

Ocrelizumab, approved for relapsing and primary progressive forms of MS, differs from rituximab in that it has a humanized antibody backbone. Ocrelizumab exhibits greater ADCC compared to CDC than rituximab and also depletes B cells through multiple mechanisms, including apoptosis and antibody-dependent cellular phagocytosis.

Ofatumumab, a fully human monoclonal antibody previously approved for refractory chronic lymphocytic leukemia, results in greater CDC than ADCC activity and is the only anti-CD20 mAb now being tested using a subcutaneous, rather than intravenous, dosing regimen. US Food and Drug Administration (FDA) has recently approved Kesimpta® (ofatumumab, formerly OMB157) as an injection for subcutaneous use for the treatment of relapsing forms of multiple sclerosis (RMS), to include clinically isolated syndrome, relapsing-remitting disease, and active secondary progressive disease, in adults. Similar, European Commission has recently approved Kesimpta® for the treatment of relapsing forms of multiple sclerosis (RMS) in adults with active disease defined by clinical or imaging features. Kesimpta is a targeted, precisely dosed and delivered B-cell therapy that has shown superior efficacy with a similar safety profile compared with teriflunomide, a first-line treatment in MS.

Other anti-CD20 mAbs include obinutuzumab, a humanized IgG1 targeting partly the same epitope of CD20 as rituximab, but designed to induce greater cell death due to its on/off binding rates, and ublituximab, an anti-CD20 antibody glycoengineered for higher affinity to all Fcγ RIIIa receptors, yielding greater ADCC than rituximab and ofatumumab, especially in cells with low CD20 expression.

However, treatment with B cell-depleting therapies has been reported to result in a higher risk of serious infectious events (SIEs) in patients recruited in Asia than were those recruited outside of Asia (Emery P, Rigby W, Tak P P, Do\ddot{ }rner T, Olech E, et al. (2014) Safety with Ocrelizumab in Rheumatoid Arthritis: Results from the Ocrelizumab Phase III). Harigai et al. suggested that treatment with ocrelizumab in Japanese patients with RA may have higher risk for Pneumocystis jirovecii pneumonia (PCP) than treatment with other biologic agents (Harigai et al., The Journal of Rheumatology 2012; 39:3; doi: 10.3899/jrheum.110994). In this context, it must be noted that treatment with B cell-depleting therapies (such as ocrelizumab) has also been reported to result in decreased serum levels of immunoglobulin IgG, IgM and/or IgA, see for example presentation of Dr. B. Wildemann at “8. Heidelberger Patiententag”, Jan. 25, 2020. Decreased IgG levels were reported to increase the risk of infections by a factor of three, decreased IgM levels were reported to double the risk of infections. In this regard it has to be kept in mind that MS therapy usually is a life-long therapy.

For example, under therapy with RTX (rituximab), patients experienced a significant time-dependent decrease of IgG and IgM levels in serum (IgG: p=2.2×10⁻⁵, IgM: p=4.0×10⁻⁴). Overall IgG levels decreased by 5.1% per year, IgM levels by 5.0%, see Klein et al., ECTRIMS Online Library, 09/13/19; 278658; P1618.

T. Derfuss et al. (“Serum immunoglobulin levels and risk of serious infections in the pivotal Phase III trials of ocrelizumab in multiple sclerosis and their open-label extensions”, ECTRIMS Online Library. Derfuss T. 09/11/19; 279399; 65) assessed serum Ig levels over 5.5 years. They observed a reduction in serum Ig levels, with an apparent association with increased rates of serious infections. The association was strongest for IgG and less so for IgM or IgA. The reduction in serum Ig levels proceeded at an approximate mean rate of 3-4% per year (see FIG. 5). There was an apparent association between decreased levels of IgG and serious infections.

Further, the ocrelizumab prescribing information describes the association between the decrease in immunoglobulins and serious infections as follows: “Treatment with Ocrevus resulted in a decrease in total immunoglobulins over the controlled period of the studies mainly driven by reduction in IgM. Clinical trial data have shown an association between decreased levels of IgG (and less so for IgM or IgA) and serious infections.” Ocrevus has not been approved so far in important Asian countries like China, Japan and Korea.

Taken together, there is a special risk of adverse events associated with B cell-depleting therapies such as ocrelizumab therapy in Asian patients. In the long term, there is a need to reduce this risk.

Therefore, the problem underlying the present invention is to provide improved treatment strategies for Asian MS patients, especially for long-term treatments. In particular, it was an object of the present invention to provide a B cell-depleting MS therapy without undesirably putting Asian patients at risk.

The problem has unexpectedly been solved by the administration of ofatumumab.

It is completely surprising that ofatumumab therapy is advantageous compared to other B cell-depleting therapies because it does not cause an increased risk of serious infections in Asians or patients having an Asian genetic background, e.g. patients of Asian phenotype. Consequently, the absence or amelioration of negative effects opens up new avenues for patients under long-term treatment. This is an important clinical benefit which is explained in detail hereinafter.

In this regard it is noted that ofatumumab surprisingly does not cause reduction of immunoglobulins (e.g. IgG) in the extent known from other B cell-depleting therapies known in the art. Such an unexpected absence of negative effects on the immune system allows treating specific patient groups, e.g. patient groups being regarded as difficult to treat, in particular in view of a long-term treatment.

Hence, a subject of the present invention relates to ofatumumab for use in the treatment of multiple sclerosis, wherein patients are treated who are of Asian race.

In a further subject of the present invention, patients are treated who have a genetic background related to Asian race, in particular who are of Asian phenotype. As used herein, the term Asian phenotype relates to people who
harbor the CYP 2C9*2 allele of cytochrome P450 (CYP) at a frequency of less than 10%, preferably less than 5%, more preferably less than 4.5%, even more preferably less than 4.4%, most preferably less than 4%, and/or
have epidermal growth factor receptor (EGFR) mutations and/or
have the VKORC1 low warfarin dose haplotype and/or
possess the promyelocytic leukemia protein (PML) gene breakpoint cluster region-1 subtype (bcr1) of chromosomal translocation t(15:17).

Thus, the present invention provides personalized or precision medicine for patients of Asian phenotype. Factoring in Asian phenotypes is essential for the therapeutic success of MS treatment and the development of improved clinical practice.

Generally, certain physical attributes distinguish Asians from Westerners. Asians generally tend to be smaller in body frame, based on the wrist circumference or elbow width, compared with Westerners when controlled for the same age and gender. Complementing observations of overt differences in terms of physique, studies have confirmed that the average height and weight of the Asian population are generally lower than their Westerners counterparts. The health implications of these and other differences are crucial because many disease thresholds are significantly different when Asians are compared to other populations for a given range of anthropometric indices when adjusted for age and sex.

Asian-prevalent diseases are those with a high disease burden and exhibiting differences in prevalence in Asia relative to the Western countries. Epidemiological data from healthcare organizations and disease registries reveal insights into etiology and disease biology, genetic predilection or uniqueness of those diseases in Asians. A number of conditions, such as nasopharyngeal carcinoma, Brugada syndrome and thyrotoxic periodic paralysis strikingly affect Asians compared to non-Asians. Therefore, a further subject of the present invention relates to ofatumumab for use in the treatment of multiple sclerosis, wherein the patients
have neuromyelitis optica (NMO) and/or
have NMO spectrum disorders and/or
have a history of sudden death (Brugada syndrome) in their family and/or
have cardiac sodium channelopathy from SCN5A loss-of-function mutation with arrthymogenic susceptibility,
have thyrotoxic periodic paralysis and/or
are obese (preferably central obesity characterized by a waist circumference >80 cm in women and >90 cm in men, more preferably combined with a “thin outside fat inside (TOFI)” characteristic phenotype of obesity) and/or have type 2 diabetes mellitus and/or
have diabetic nephropathy and/or
have aggressive triple negative or basal breast cancer (ER, PR, Her2/neu negative) and/or
have a history of lacunar strokes and/or
have a history of intracerebral haemorrhage and/or
I have systemic lupus erythematosus (SLE) and/or
suffer from obstructive sleep apnea (OSA) and/or
have nasopharyngeal cancer (NPC), preferably differentiated non-keratinizing carcinoma (WHO Type 2 histology) and/or
have insulin resistance.

A further subject of the present invention is ofatumumab for use in the treatment of multiple sclerosis, wherein the treatment is ethnically insensitive.

The ethnic insensitivity of ofatumumab may comprise on or more of the following features:
As a fully human IgG1 monoclonal antibody (mAb), the pharmacokinetics (PK) of ofatumumab was demonstrated to be ethnically insensitive:
   Comparable ofatumumab concentrations in Asian subjects and White subjects have been observed in pivotal Phase III studies in patients with RMS.
   Race (White, Black, Asian, unknown, other) was tested as a covariate in the population PK analysis of PK data in patients with MS and was not found to be a significant covariate impacting the PK of ofatumumab.
   Comparable PK parameters (Cmax, AUC, T_{1/2}) following 2000 mg intravenous (i.v.) infusion administration were observed in Asian patients (9 Japanese, and 1 Korean) vs. in White (96% of the patients are Whites) patients with refractory CLL.
   The principal route of clearance for low-dose ofatumumab after the first injection is via target-mediated clearance by CD20 binding and B-cell lysis. Similar CD20+ B-cell baseline levels were observed between Asian and non-Asian patients with RMS. Therefore, the target-mediated clearance of ofatumumab in Asian patients with RMS is comparable with that in non-Asian patients with RMS.
   The PK exposure was consistent with observations in previous studies and led to a rapid and consistent depletion of CD19+ B-cells and CD3+CD20+ T-cells indicating similar pharmacodynamic response in ofatumumab treated patients in Japan and non-Japan subgroups.
   The principal route of clearance for low-dose ofatumumab during the maintenance treatment is expected to be mediated by nonspecific catabolismpathways. The catabolismby ubiquitous proteolytic enzymes is not expected to be ethnically sensitive as demonstrated by many other mAb products.
A comparable efficacy profile was established between Asian and non-Asian patients in RMS:
   The efficacy profile in Asian subgroups (by race and region) was consistent with the overall efficacy profile of ofatumumab as demonstrated in pivotal Phase III studies.
   Results from a Phase II study demonstrated superior efficacy of ofatumumab treatment vs. placebo in lesion suppression, with consistently lower lesion numbers in the ofatumumab group in both Japan and non-Japan subgroups of patients.
A comparable safety profile was established between Asian and non-Asian patients with RMS:
   No new safety concerns were identified in the Asian subgroups (by race and region) and the overall safety profile of ofatumumab is favourable as demonstrated in the Phase III pivotal studies in patients with RMS. The studies included a total number of 71 Asian patients (by race) with RMS exposed to ofatumumab.
   Results from a Phase II study including Japan and non-Japan subgroups of patients with RMS showed that no new safety signals were detected and that the safety profile was in line with that observed in the global pivotal studies.

### DESCRIPTION OF PREFERRED EMBODIMENTS

Generally, the present invention concerns the treatment of multiple sclerosis. In a preferred embodiment the invention relates to the treatment of relapsing multiple sclerosis (RMS). In particular, the present invention relates to the treatment of relapsing remitting multiple sclerosis (RRMS). Alternatively, the present invention relates to the treatment of secondary progressive MS (SPMS). Further alternatively, the present invention relates to the treatment of clinically isolated syndrome (CIS). Again further alternatively, the present invention relates to the treatment of primary progressive multiple sclerosis (PPMS) or progressive relapsing multiple sclerosis (PRMS).

Generally, according to the present invention ofatumumab can be administered to all MS patients, irrespective of their race, ethnicity and/or phenotype.

Generally, the patient does not have to be naïve, i.e. it is possible that the patient, preferably the Asian patient, has been treated with a disease-modifying treatment (DMT) of multiple sclerosis other than ofatumumab (such as glatiramer acetate, cladribine, fingolimod, siponimod, natalizumab, teriflunomide, mitoxantrone or dimethyl fumarate), prior to commencing ofatumumab treatment.

In a first aspect of the present invention, patients are treated with ofatumumab who are of Asian race. Patients of Chinese, Japanese or Korean race may be preferred.

In an alternative embodiment, patients are treated who
harbor the CYP 2C9*2 allele of cytochrome P450 (CYP) at a frequency of less than 10%, preferably less than 5%, more preferably less than 4.5%, even more preferably less than 4.4%, most preferably less than 4%, and/or
have epidermal growth factor receptor (EGFR) mutations and/or
have the VKORC1 low warfarin dose haplotype and/or
possess the promyelocytic leukemia protein (PML) gene breakpoint cluster region-1 subtype (bcr1) of chromosomal translocation t(15:17).

These patients preferably may happen to be of Asian race.

Generally, the above-identified alleles, genotypes and mutations can be detected by standard procedures. Examples include but are not limited to Conventional Karyotyping, Fluorescence in situ hybridization (FISH), Comparative genomic hybridization (CGH) and Next-Generation Sequencing. Methods for use in the present invention have been described in Iran J Pediatr. 2013 August; 23(4): 375-388 (PMCID: PMC3883366; PMID: 24427490).

In an embodiment of the present invention, ofatumumab is used if there are indicators of a risk of MS therapy-affecting co-morbidities e.g. infections, such as reduced serum IgG levels. In other words, if serum IgG levels are undesirably low, treatment of MS is carried out or continued with ofatumumab as the DMT. In a preferred embodiment of the present invention, ofatumumab is administered as the sole active ingredient for treating MS, i.e. the only disease-modifying drug that is administered.

Other indicators of a risk of MS therapy-affecting co-morbidities include
neuromyelitis optica (NMO),
NMO spectrum disorders,
a history of sudden death (Brugada syndrome) in their family, cardiac sodium channelopathy from SCN5A loss-of-function mutation with arrthymogenic susceptibility,
thyrotoxic periodic paralysis,
obesity (preferably central obesity characterized by a waist circumference >80 cm in women and >90 cm in men, more preferably combined with a “thin outside fat inside (TOFI)” characteristic phenotype of obesity),
type 2 diabetes mellitus,
diabetic nephropathy,
aggressive triple negative or basal breast cancer (ER, PR, Her2/neu negative),
a history of lacunar strokes,
a history of intracerebral hemorrhage,
systemic lupus erythematosus (SLE),
obstructive sleep apnea (OSA),
nasopharyngeal cancer (NPC), preferably differentiated non-keratinizing carcinoma (WHO Type 2 histology), and/or
insulin resistance.

Therefore, the present invention also relates to ofatumumab for use in the treatment of multiple sclerosis, wherein the patient, preferably the Asian patient, has or suffers from one of the above-identified conditions.

In a second aspect of the present invention, ofatumumab is used in the treatment of multiple sclerosis, wherein the treatment is ethnically insensitive.

In a preferred embodiment of the present invention, ofatumumab is administered as the sole active ingredient for treating MS, i.e. the only disease-modifying drug that is administered.

As mentioned above, a subject of the present invention is ofatumumab for use in the treatment of multiple sclerosis, wherein the treatment is a long-term treatment and wherein the treatment is ethnically insensitive.

In a preferred embodiment, the adverse events occurring in patients of Asian race are consistent with the overall safety profile of ofatumumab, i.e. comparable with the safety profile in all other patients being not of Asian race.

In a preferred embodiment, the adverse events are selected from injection-related reactions, infections, malignant and premalignant disorder, hepatic damage or dysfunction and neutropenia.

In an alternative preferred embodiment, the infections are selected from respiratory tract infections, urinary tract infections, Herpes viral infections, Varicella-Zoster infections, opportunistic infections, pulmonary tuberculosis, HBV infection reactivation and progressive multifocal leukoencephalopathy (PML).

In a preferred embodiment, the infections are serious infections, preferably selected from opportunistic infections, HBV infection reactivation, Pneumocystis jirovecii pneumonia (PCP) and PML.

For example, an increased number of malignancies (including breast cancers) have been observed in clinical trials in patients treated with ocrelizumab (Ocrevus®), compared to control groups. Ocrevus' SmPC recommends that individual benefit/risk should be considered in patients who are being actively monitored for recurrence of malignancy. Patients with a known active malignancy should not be treated with Ocrevus.

As mentioned above, side effects and adverse events associated with B cell-depleting therapies such as ocrelizumab therapy are reported to be associated with a reduction of immunoglobulins (e.g. IgG). In the present invention it was surprisingly found that ofatumumab therapy is advantageous compared to other B cell-depleting therapies because it does not cause reduction of immunoglobulins (e.g. IgG) in the long run and thus opens up new avenues for patients under long-term treatment.

Hence, in a preferred embodiment of the present invention ofatumumab is used in the treatment of MS, wherein ofatumumab is administered to patients, preferably Asian patients, with known risk factors for malignancies. In another preferred embodiment of the present invention ofatumumab is used in the treatment of MS, wherein ofatumumab is administered to patients, preferably Asian patients, who are being actively monitored for recurrence of malignancy. In an alternative embodiment of the present invention ofatumumab is used in the treatment of MS, wherein ofatumumab is administered to patients, preferably Asian patients, with a known active malignancy.

In a preferred embodiment of the present invention, the patients are switched from an earlier disease modifying treatment (DMT) to ofatumumab, wherein the switch is preferably made when there is a change in
the earlier DMT's Cmax, and/or
the earlier DMT's AUC, and/or
B cell and/or T cell inhibition or depletion, and/or
serum IgG level, and/or
adverse events.

In a preferred embodiment of the present invention, serum IgG levels are maintained during the treatment within a range from 500 to 1800 mg/dl. In a preferred embodiment, ofatumumab is used if the serum IgG level falls below a concentration of 900 mg/dl, 850 mg/dl, 800 mg/dl, 750 mg/dl, 700 mg/dl, 650 mg/ml, 600 mg/dl, 550 mg/dl or 500 mg/dl.

In a preferred embodiment, ofatumumab is used if the serum IgG level falls below the lower limit of normal, hereinafter referred to as “LLN”. Generally, the lower limit of normal (LLN) for IgG, IgA and IgM can be defined as IgG=700 mg/dl or 565 mg/dl, IgM=40 mg/dl and IgA=70 mg/dl.

In a preferred embodiment of the present invention, ofatumumab is used in the treatment of MS, wherein the treatment is a long-term treatment. The term long-term treatment indicates that ofatumumab is used over an extended period of time. For example, ofatumumab can be used for more than 2 years, 3 years, 4 years, 5, years, 10 years. Ofatumumab might be used up to 5 years, 10 years, 15 years, 20 years or for life.

In a preferred embodiment of the present invention, ofatumumab is administered at a dose of 10 to 30 mg every 4 weeks, preferably 20 mg every 4 weeks. Preferably, ofatumumab is administered parenterally, e.g. by epidermal, intravenous, intramuscular, intraarterial, intrathecal, intracapsular, intraorbital, intra-cardiac, intradermal, intraperitoneal, intratendinous, transtracheal, subcutaneous, subcuticular, intraarticular, subcapsular, subarachnoid, intraspinal, intracranial, intrathoracic, epidural or intrasternal injection or infusion. The preferred route of administration is a subcutaneous injection (sc).

In a preferred embodiment of the present invention, ofatumumab is administered with a loading dose. The term loading dose is defined below. In a preferred embodiment, three loading doses are administered, preferably in week 0 and in week 1 and in week 2 after starting ofatumumab therapy. This means the first loading dose in week 0 constitutes the start of therapy. In an alternative preferred embodiment, three loading doses are administered on day 1, on day 5-9, preferably on day 7, and on day 12-16, preferably on day 14, after starting ofatumumab therapy. This means the first loading dose on day 1 constitutes the start of therapy.

In a preferred embodiment of the present invention, a loading dose is 10-30 mg, preferably 20 mg ofatumumab at weeks 0, 1 and 2.

In an alternative embodiment of the present invention, ofatumumab is administered without a loading dose.

In a preferred embodiment of the present invention, a premedication is administered to the patient before the first dose of ofatumumab is administered. Preferably, the premedication comprises a compound selected from acetaminophen, antihistamines and steroids. Methylprednisolone may be a preferred steroid. 100 mg iv may be a preferred dose. Preferably, the premedication is administered 30 to 60 minutes prior to an ofatumumab injection.

In a particular preferred embodiment of the present invention, no premedication is administered prior to the first dose of ofatumumab.

In a preferred embodiment of the present invention, relapsing multiple sclerosis is either clinically isolated syndrome (CIS) or relapsing remitting multiple sclerosis (RRMS) or secondary progressive multiple sclerosis (SPMS). These terms are defined below.

In a preferred embodiment of the present invention, multiple sclerosis is selected from primary progressive multiple sclerosis (PPMS) or progressive relapsing multiple sclerosis (PRMS).

In a preferred embodiment of the present invention, ofatumumab is administered as the sole active ingredient for treating MS. In other words, ofatumumab is preferably the only disease-modifying drug that is administered.

In a preferred embodiment, ofatumumab can be administered irrespective of body weight, sex, age, race or baseline B-cell count. For example, it is preferred that a 35-year-old woman having a body weight of 60 kg receives the same dose as a 50-year old man having a body weight of 90 kg. In particular, body weight, sex, age, race or baseline B-cell count do not have a clinically meaningful effect on the pharmacokinetics of ofatumumab.

The MSIS-29 (see definition below) is a clinically useful and scientifically sound measure of the impact of MS from the patient's perspective suitable for clinical studies and epidemiological studies. It is considered a reliable, valid and responsive PRO (Patient Reported Outcomes) measure that complements other indicators of disease severity used to improve our understanding of the impact of MS.

In the present invention, it was unexpectedly found that administration of ofatumumab to Asian patients leads to an advantageous reduction of the MS impact scale MSIS-29 as defined below.

In this regard a further subject of the present invention is ofatumumab for use in the treatment or prevention of relapsing multiple sclerosis, wherein ofatumumab reduces the MSIS-29 score, preferably in Asian patients. Preferably, ofatumumab reduces the MSIS-29 score by at least 1.5, more preferably at least 2.0, still more preferably at least 2.5 within 24 months. The reduction might be up to 3.0 or 3.5 or 4.0.

In one embodiment of the invention, an ofatumumab composition is formulated in accordance with routine procedures as a pharmaceutical composition adapted for intravenous administration to human beings. Typically, compositions for intravenous administration are solutions in sterile isotonic aqueous buffer. Where suitable, the composition may also include a solubilizing agent and a local anesthetic, such as lignocaine, to ease pain at the injection site. Generally, the ingredients are supplied either separately or mixed together in a unit dosage form, for example as a dry lyophilized powder or water-free concentrate, in a hermetically sealed container, such as an ampoule or sachet, indicating the quantity of active agent.

Where the composition is to be administered by infusion, in particular by subcutaneous injection (s.c.), it can be dispensed with an infusion bottle containing sterile pharmaceutical grade water or saline.

Where the composition is administered by injection, an ampoule of sterile water for injection or saline can be provided so that the ingredients may be mixed prior to administration.

In one embodiment, a formulation for ofatumumab can be formulated according to a formulation disclosed in WO 2009/009407.

In one embodiment, ofatumumab is formulated in an antibody formulation wherein ofatumumab is present in an amount of about 20-300 mg/mL, 50-300 mg/mL, 100-300 mg/mL, 150-300 mg/mL, 200-300 mg/mL or 250-300 mg/mL, preferably at 50 mg/ml.

In one embodiment, ofatumumab is formulated in an antibody formulation wherein the formulation comprises 10 to 100 mM sodium acetate, 25 to 100 mM sodium chloride, 0.5 to 5% arginine free base, 0.02 to 0.2 mM EDTA, 0.01 to 0.2% polysorbate 80 and adjusted to pH 5.0 to 7.0. Preferably, the ofatumumab formulation comprises 50 mM sodium acetate, 51 mM sodium chloride, 1% arginine free base, 0.05 mM EDTA, 0.02% polysorbate 80 and adjusted to pH 5.5.

The preferred dosage of ofatumumab is:
initial dosing of 20 mg by subcutaneous injection at Weeks 0, 1 and 2 (or on day 1, on day 5-9, preferably on day 7, and on day 12-16, preferably on day 14), followed by
subsequent dosing of 20 mg by subcutaneous injection once monthly starting at Week 4.

If an injection of ofatumumab is missed, it should preferably be administered as soon as possible without waiting until the next scheduled dose. Subsequent doses should be administered at the recommended intervals.

In one embodiment, the ofatumumab formulation is provided in a pre-filled syringe or in an auto-injector, preferably a single-dose pre-filled syringe or a single-dose pre-filled auto-injector. Preferably, a pre-filled auto-injector designed for s.c. administration is used.

In a preferred embodiment, ofatumumab injection is a sterile, preservative-free solution for subcutaneous use. Preferably, each 20 mg/0.4 mL prefilled pen or prefilled syringe delivers 0.4 mL of solution. Preferably, each 0.4 mL contains 20 mg of ofatumumab and arginine (4 mg), disodium edetate (0.007 mg), polysorbate 80 (0.08 mg), sodium acetate trihydrate (2.722 mg), sodium chloride (1.192 mg) and Water for Injection, USP with a pH of 5.5. Hydrochloric acid may be added to adjust pH.

In a preferred embodiment the ofatumumab formulation is intended for patient self-administration, preferably by subcutaneous injection.

In a preferred embodiment said formulation is administered in the abdomen, thigh or outer upper arm subcutaneously. In a preferred embodiment said formulation is not administered into moles, scars or areas where the skin is tender, bruised, red, hard or not intact.

In an embodiment, the first injection of said ofatumumab formulation may be performed under the guidance of a healthcare professional. If injection-related reactions occur, symptomatic treatment is recommended. Before administration, the pen or prefilled syringe is preferably removed from the refrigerator and allowed to reach room temperature, e.g. for about 15 to 30 minutes. In a preferred embodiment, the ofatumumab formulation of the present invention is a clear to slightly opalescent and colorless to slightly brownish-yellow solution available as follows:
Injection: 20 mg/0.4 mL in a single-dose prefilled pen, e.g. Sensoready® pen
Injection: 20 mg/0.4 mL in a single-dose prefilled syringe.

In a preferred embodiment, a subcutaneous ofatumumab dose of 20 mg every 4 weeks leads to a mean AUCₜₐᵤ of about 400 to 550, more preferably 450 to 500, e.g. 483 mcg h/mL and/or to a mean Cₘₐₓ of 1.0 to 2.5, more preferably 1.2 to 1.7, e.g. 1.43 mcg/mL at steady state. In a preferred embodiment, the volume of distribution at steady-state can be 4.5 to 6.5, more preferably 5.0 to 6.0, e.g. 5.42 L following subcutaneous administration of repeated ofatumumab 20 mg doses.

After subcutaneous administration, ofatumumab can be absorbed via the lymphatic system.

In a preferred embodiment of the invention, ofatumumab administration is delayed in patients with an active infection, e.g. COVID-19, until the infection is resolved. Thus, the treatment is interrupted during COVID-19 infection und continued after overcoming the infection.

Alternatively, the ofatumumab can be administered during an infection, e.g. during a COVID-19 infection. Thus, ofatumumab administration can be continued during an infection, e.g. during a COVID-19 infection. In particularly preferred embodiment, a patient acutely or previously infected by COVID-19 is treated with ofatumumab.

In another preferred embodiment of the invention, the level of immunoglobulins at the beginning, during and after discontinuation of treatment with ofatumumab are monitored as clinically indicated until B-cell repletion. Discontinuing ofatumumab treatment is considered if a patient develops a serious opportunistic infection or recurrent infections if immunoglobulin levels indicate immune compromise.

A further subject of the invention is a method for treating multiple sclerosis, said treatment comprising administering ofatumumab to a patient in need thereof, wherein the patient is of Asian race.

A further subject of the invention is a method for treating multiple sclerosis, said treatment comprising administering ofatumumab to a patient in need thereof, wherein the treatment is ethnically insensitive.

A further subject of the present invention is a method for the manufacture of a medicament for use in the treatments described above.

All preferred embodiments described above generally apply to these subjects.

### Definitions

Race may be defined as the descendants of a common ancestor, or a group of people with distinct physical and genetic traits or characteristics that are passed on through birth.

Asian race may comprise persons with ancestors in any of the original peoples of the Far East, the Indian subcontinent including Cambodia, China, India, Japan, Korea, Malaysia, Pakistan, Philippine Islands, Thailand, Vietnam, Hmong, East India, Laos, Bangladesh, Indonesia, Sri Lanka, Nepal, Bhutan, Sikh, Burma and other South and Southeast Asian.

Chinese—Includes persons who indicated their race as Chinese, or who identified themselves as Cantonese, Tibetan, or Chinese American. In standard census reports, persons who reported as Taiwanese or Formosan are included here with Chinese.

Filipino—Includes persons who indicated their race as Filipino, Pilipino, or Philipine.

Japanese—Includes persons who indicated their race as Japanese, Nipponese or Japanese American.

Korean—Includes persons who indicated their race as Korean or Korean American

Vietnamese—Includes persons who indicated their race as Vietnamese or Vietnamese American.

Other Southeast Asian—Includes persons from one of the Southeast Asian countries or groups including Laos, Hmong, Laohmong, Mong, Cambodia, Thailand, Siamese, Malaysia.

South Asian—Includes persons from one of the South Asian countries including Afghanistan, India, Pakistan, Bangladesh, Nepal and Sri Lanka.

Other Asian—Includes persons from or considering themselves to be Burmese, Indonesian, Bengali, Bharat, Dravidian, East India, Goanese or Asian Indian.

Caucasian refers to the ‘white race’ of Mankind derived from the region of the Caucasus Mountains in Europe. Presently, the United States National Library of Medicine has discontinued this racial term as antiquated in favor of the term ‘European’. For the sake of this application, the term Westerners is adopted to encompass the Europeans and the white race of the United Kingdom and North America.

The term ethnically insensitive preferably means that there are no therapeutically relevant differences between different races, ethnicities or phenotypes. The ethnic insensitivity of ofatumumab particularly relates to ofatumumab having
ethnically insensitive pharmacokinetics (PK),
a comparable efficacy profile between Asian and non-Asian patients in RMS,
a comparable safety profile between Asian and non-Asian patients with RMS.

The terms “treatment” or “treat” can be defined as the application or administration of e.g. ofatumumab to a patient, where the purpose is to abolish, reduce or alleviate the symptoms of a disease such as multiple sclerosis (MS). In particular, the term “treatment” comprises the achievement of a clinically meaningful effect for the patient, for example the achievement of a clinically meaningful reduction of the annual relapse rate when treating RMS.

As used herein, a patient can be “in need of” a treatment if such a patient would benefit medically or in terms of the quality of life from such treatment.

The term “patient” as used herein can refer to a mammal, e.g. a primate, preferably a higher primate, especially preferred a human (e.g. a patient having a risk or at risk of having a disorder described herein). Preferably, the patient is an adult. Generally, also geriatric patients are included, however, patients between 18 and 60 years of age are preferred. As used herein, the term “administering” or “administration” of ofatumumab can mean providing ofatumumab to a patient in need of treatment. Administration “in combination with” one or more further therapeutic agents includes simultaneous (concurrent) and consecutive administration in any order and in any route of administration.

As used herein, a “therapeutically effective amount” can refer to an amount of ofatumumab that is effective, i.e. achieves a clinically meaningful effect.

The term “adverse event” (AE) can relate to any untoward medical occurrence in a patient or clinical investigation wherein the subject is administered a pharmaceutical product which does not necessarily have a causal relationship with this treatment. An adverse event (AE) can therefore be any unfavourable and unintended sign (including an abnormal laboratory finding), symptom or disease temporally associated with the use of a medicinal (investigational) product, whether or not related to the medicinal (investigational) product.

The phrase “therapeutic regimen” can mean the regimen used to treat an illness or to prevent a disease condition or the development of a disease, e.g. the dosing used. A therapeutic regimen may include an induction regimen, a loading regimen and a maintenance regimen.

### RRMS

Relapsing-remitting multiple sclerosis (RRMS) may be characterized by relapses, defined as a new neurologic deficit or episode of neurologic worsening lasting longer than 24 h, preferably in the absence of fever or infection.

There may be no apparent progression of the disease during the periods of remission. At different points in time, RRMS might be further characterized as either active (with relapses and/or evidence of new MRI activity) or not active, as well as worsening (a confirmed increase in disability over a specified period of time following a relapse) or not worsening. Reference is made to Lublin, Neurology. 2014 Jul. 15; 83(3): 278-286.

### RMS

The term RMS (relapsing multiple sclerosis) encompasses RRMS, SPMS and clinically isolated syndrome (CIS).

### Primary Progressive MS (PPMS)

PPMS can be characterized by worsening neurologic functions (accumulation of disability) from the onset of symptoms, without early relapses or remissions. PPMS can be further characterized at different points in time as either active (with an occasional relapse and/or evidence of new MRI activity) or not active, as well as with progression (evidence of disease worsening on an objective measure of change over time, with or without relapse or new MRI activity) or without progression. References is made to Lublin 2014.

Each person's experience with PPMS will be unique. PPMS can have brief periods when the disease is stable, with or without a relapse or new MRI activity, as well as periods when increasing disability occurs with or without new relapses or lesions on MRI.

### Secondary Progressive MS (SPMS)

SPMS follows an initial relapsing-remitting course. Most people who are diagnosed with RRMS will eventually transition to a secondary progressive course in which there is a progressive worsening of neurologic function (accumulation of disability) over time. SPMS can be further characterized at different points in time as either active (with relapses and/or evidence of new MRI activity) or not active, as well as with progression (evidence of disease worsening on an objective measure of change over time, with or without relapses) or without progression. Reference is made to Lublin 2014.

Each person's experience with SPMS will be unique. SPMS follows after relapsing-remitting MS. Disability gradually increases over time, with or without evidence of disease activity (relapses or changes on MRI). In SPMS, occasional relapses may occur, as well as periods of stability.

### Relapse

Relapses can be defined as a new neurologic deficit or episode of neurologic worsening, preferably lasting longer than 24 h. In other words, relapses can be regarded as discrete episodes (in the art also referred to as “attacks,” “flare-ups” or “exacerbations”) of neurologic dysfunction, preferably lasting at least 24 h. Usually, relapses are followed by full or partial recovery and a period in which there is no symptom progression or accumulation of disability (remission).

“B cell inhibitor” as used herein generally may relate to any substance that abolishes, reduces or attenuates biological B cell functions. The B cell inhibitor may interrupt signal transduction pathways that are necessary for biological B cell functions, e.g. cytokine secretion or responses to cis and/or trans stimulation. The B cell inhibitor may also interfere with the generation of B cells from stem/progenitor cells or negatively affect their maturation. Moreover, the B cell inhibitor may act by inhibiting the cross-talk with other cell populations such as T cells. Alternatively, the B cell inhibitor may deplete B cells by sequestration (e.g. into lymphoid tissues such as the spleen) or by lysis e.g. through CDC, ADCC, phagocytosis or other processes. Several subsets of B cells may express CD20.

B cell as used herein may relate to a type of white blood cell of the lymphocyte subtype. B cells function in the humoral immunity component of the adaptive immune system by secreting antibodies such as immunoglobulins (e.g. IgG). Additionally, B cells may present antigens and secrete cytokines. B cells, unlike the T cells and natural killer cells, express B cell receptors (BCRs) on their cell membrane. BCRs allow the B cell to bind to a specific antigen against which it will initiate an antibody response.

In a preferred embodiment of the present invention the use of ofatumumab maintains an IgG level which is essentially in the same range as in untreated patients, preferably as in the untreated Asian patients. In the context of the present invention, “untreated patients” refers to patients diagnosed with MS or clinically isolated syndrome (CIS) and which are not administered a B cell and/or T cell inhibitor. In a preferred embodiment the untreated patients present IgG levels in a range from 500 to 1800 mg/dl, particularly from 700 to 1600 mg/dl, more particularly from 900 to 1400 mg/dl. Particularly, the untreated patients present IgG levels from 500 mg/dl, 550 mg/dl, 600 mg/dl, 650 mg/dl, 700 mg/dl, 750 mg/dl, 800 mg/dl, 850 mg/dl, 900 mg/dl up to 1400 mg/dl, 1500 mg/dl, 1600 mg/dl, 1700 mg/dl, 1800 mg/dl.

### Clinically Isolated Syndrome (CIS):

Clinically isolated syndrome (CIS) may refer to a single clinical attack of central nervous system (CNS) inflammatory demyelinating symptoms that are suggestive of multiple sclerosis (MS). CIS presentations can be monofocal or multifocal and typically may involve the optic nerve, brainstem, cerebellum, spinal cord or cerebral hemispheres. Reference is made to Miller et al, Clinically isolated syndromes, Lancet Neurol. 2012; 11:157-169.

### Multiple Sclerosis Impact Scale (MSIS-29)

The MSIS-29 version 2 is a 29-item, self-administered questionnaire that includes 2 domains: physical and psychological. Responses were captured on a 4-point ordinal scale ranging from 1 (not at all) to 4 (extremely), with higher scores reflecting greater impact on day-to-day life. The MSIS-29 takes about 5 minutes to complete and the questions are designed to determine the patient's views about the impact of MS on their day-to-day life during the past 2 weeks. Reference is made to Hobart J and Cano S (2009), “Improving the evaluation of therapeutic interventions in multiple sclerosis: the role of new psychometric methods”, Health Technol Assess; 13(12):iii, ix-x, 1-177. NS RO to Hobart J, Lamping D, Fitzpatrick R, et al (2001), “The Multiple Sclerosis Impact Scale (MSIS-29): a new patient-based outcome measure”, Brain; 124(Pt 5): 962-73.

### Ofatumumab:

Ofatumumab is a human monoclonal antibody for the CD20 protein. Ofatumumab may bind specifically to both the small and large extracellular loops of the CD20 molecule. The Fab domain of ofatumumab may bind to the CD20 molecule and the Fc domain mediates immune effector functions to result in B-cell lysis in vitro. In particular, ofatumumab is a recombinant human monoclonal immunoglobulin G1 (IgG1) antibody that binds to human CD20 expressed on e.g. B cells. Ofatumumab is produced in a murine NS0 cell line and consists of two IgG1 heavy chains and two kappa light chains with a molecular weight of approximately 146 kDa.

Ofatumumab is described in EP 1 558 648 B1 and EP 3 284 753 B1. Further reference is made to the description in the drugbank.ca, accession number DB06650 and to WHO Drug Information, Vol. 20, No. 1, 2006. In an embodiment, the protein chemical formula is C₆₄₈₀H₁₀₀₂₂N₁₇₄₂O₂₀₂₀S₄₄ and the protein average weight is about 146100 Da. In US and EP ofatumumab is marketed under the tradename Kesimpta®.

The metabolic pathway of ofatumumab can be degradation to small peptides and amino acids by ubiquitous proteolytic enzymes. Ofatumumab might be eliminated in two ways: a target-independent route as with other IgG molecules and a target-mediated route that is related to binding to B cells.

The half-life of ofatumumab at steady state can be approximately 16 days, in particular following subcutaneous administration of repeated 20 mg doses. Ofatumumab preferably does not share a common clearance pathway with chemical drugs that are metabolized by the cytochrome P450 system or other drug metabolizing enzymes. Preferably, ofatumumab is not involved in the regulation of the expression of drug metabolizing enzymes.

### Loading Dose

A loading dose is an initial dose of a drug, preferably an initial higher dose, that may be given at the beginning of a treatment (e.g. a DMT) before transitioning to a maintenance dose, preferably being lower than the loading dose.

Immunoglobulins (Ig) and the subtypes IgG, IgA, IgM, IgD and IgE are commonly known and e.g. described in Berg/Tymoczko/Stryer “Biochemie”, 5th edition, pp. 1015-1018. The present application focuses on serum levels of IgG and IgM, in particular IgG.

Serum immunoglobulin (Ig) levels can be determined routinely in clinical practice. In the present application serum Ig levels can preferably be determined by immunoturbidimetry. Preferably a Roche cobas® Analyzer was used, especially module c of the cobas® analyser. In a preferred embodiment Ig levels can be determined by using a cobas® c 311 analyzer. It is further preferred that IgG measurements are carried out as described in the cobas® leaflet “IGG-2 Tina-quant IgG Gen.2”, preferably version 11.0 dated 2016-02 and IgM measurements as described in the leaflet “IGM-2 Tina-quant IgM Gen.2”, preferably version 13.0 dated 2018-11.

Serum samples were preferably kept at 2-8° C. before measurement.

### DESCRIPTION OF FIGURES

FIG. 1 is a plot of absolute values of IgG parameters by visit-window and 48, 96 and 144 weeks completers in OMB long-term group

FIG. 2 is a plot of absolute values of IgG parameters by visit-window and quartiles of baseline values in OMB long-term group

FIG. 3 is a plot of absolute values of IgM parameters by visit-window and 48, 96 and 144 weeks completers in OMB long-term group

FIG. 4 is a plot of absolute values of IgM parameters by visit-window and quartiles of baseline values in OMB long-term group

FIG. 5 shows the decrease in IgG levels following administration of an anti-CD20 antibody of the prior art (ocrelizumab). FIG. 5 was first published as part of T. Derfuss et al.: “Serum immunoglobulin levels and risk of serious infections in the pivotal Phase III trials of ocrelizumab in multiple sclerosis and their open-label extensions”, ECTRIMS Online Library. Derfuss T. 09/11/19; 279399; 65.

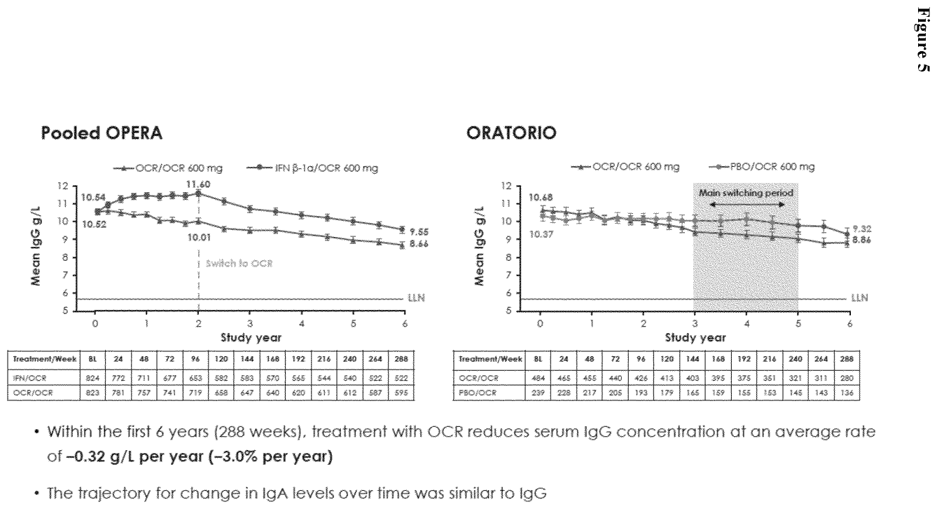

FIG. 6 shows that, after two years (96 weeks), ocrelizumab treatment has led to a reduction of IgG levels by approx. 5% whereas ofatumumab has led to an increase of about 3%.

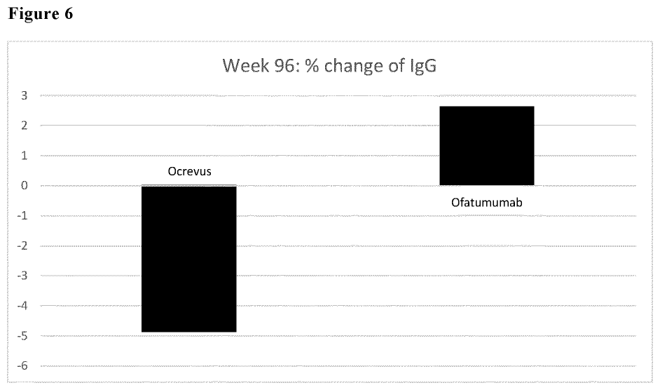

The invention may be illustrated by the following examples.

### Example 1

Ofatumumab was investigated in 1882 patients with RMS from two identical Phase III randomized, double-blind, double-dummy, active-comparator controlled, parallel-group, multicenter studies of identical design (Study I and Study II). Patients were randomized to receive either ofatumumab, OMB (20 mg s.c injections every 4 weeks, after an initial loading regimen of 3 injections on Day 1, 7, and 14), or teriflunomide, TER (14 mg p.o. once a day) as active comparator. For brevity, the teriflunomide data are not shown hereinafter.

The maximum treatment duration for an individual patient was 30 study months (approximately 2.5 years). The two studies included a total of 71 patients of Asian race (36 patients in ofatumumab and 35 patients in teriflunomide treatment groups). This Example also includes the Asian patients data from oncology studies (Study A and Study B), as supportive data, where required.

Completed RMS studies with enrollment of Asian subjects are summarized in Table 1.

TABLE 1
Tabular listing of relevant RMS clinical studies for ethnic comparisons

| | | | Ofatumumab |
|---|---|---|---|
| | | No. of subjects | treatment details |
| Study/ | | on ofatumumab | (Dose regimen; |
| Indications | Design | (Total/Asian subjects) | Route; Duration) |
| RMS Studies | | | |
| Study A | Phase II, 24-week, | 43/21 | OMB sc: 20 mg day 1, |
| | randomized, double- | | day 7, day 14 and |
| | blind, placebo-controlled, | | month 1, then 20 mg |
| | parallel group, multicenter | | every 4 weeks until |
| | study Efficacy, safety and | | 24 weeks. Subsequent |
| | PK followed by an extended | | dosing in extension |
| | treatment of at least 24 | | study will be 20 mg every |
| | weeks with open-label | | 4 weeks for 9 months |
| | ofatumumab | | |
| Study B | Phase II, 12-week, | 284/10 | OMB sc: 20 mg day 1, |
| | randomized, open-label, | | day 7, day 14 and month |
| | parallel group, multicenter | | 1, then 20 mg every 4 |
| | study Bioequivalence of | | weeks until 12 weeks. |
| | 20 mg sc ofatumumab | | Patients will be randomized |
| | injected by PFS or | | into 4 groups dependent |
| | autoinjector | | on type of injection device |
| | | | and site of injection |
| Study I | Phase III, randomized | 465/15 | OMB sc: 20 mg day 1, |
| | doubleblind, double- | | day 7, day 14 and month 1, |
| | dummy, active comparator- | | then 20 mg every 4 weeks |
| | controlled, parallelgroup | | until End of Study (max |
| | study Efficacy and safety | | 30 months) |
| | of ofatumumab vs | | |
| | teriflunomide | | |
| Study II | Phase III, randomized, | 481/21 | OMB sc: 20 mg day 1, |
| | doubleblind, double-dummy, | | day 7, day 14 and month 1, |
| | active comparator-controlled, | | then 20 mg every 4 weeks |
| | parallel group study Efficacy | | until End of Study (max |
| | and safety of ofatumumab vs | | 30 months) |
| | teriflunomide | | |

### a) Annualized Relapse Rate (ARR)

ARR was the primary efficacy endpoint in the pivotal Phase III studies. Treatment with ofatumumab and teriflunomide were both associated with relatively low ARRs. in the Asian subgroups, with 4 to 5 confirmed relapses in any of the treatment groups (ARRs of 0.08-0.09) (Table 2). In the rest-of-world (ROW) subgroups and the overall population, treatment with ofatumumab significantly reduced the adjusted ARR compared with teriflunomide by 53.3% and 52.6%, respectively (p<0.001 for all) (Table 2).

TABLE 2
Annualized relapse rates (ARR) (time-based) -
Confirmed relapses, by subgroup (FAS)

| | Number of | | | |
|---|---|---|---|---|
| | relapses/ | | | Rate |
| | patient years | | Adjusted ARR | reduction |
| | (ARR) | | (9559 | (%)/P-value |
| Asian | | | | |
| OMB (N = 36) | 4/47 | (0.084) | 0.08 (0.03, 0.24) | 11.8/0.862 |
| Rest of World | | | | |
| OMB (N = 910) | 181/1490 | (0.122) | 0.12 (0.10, 0.14) | 53.3/<0.001* |

N: Total number of patients included in the analysis. Confirmed relapses are those accompanied by a clinically relevant change in the Expanded Disability Status Scale (EDSS).
*Indicates statistical significance (2-sided) at the 0.05 level.

b) Confirmed Disability Worsening (3 mCDW, 6 mCDW)

Time to 3 mCDW and 6 mCDW were key secondary efficacy endpoints in the pivotal Phase III studies. In the overall population, treatment with ofatumumab significantly reduced the risk of both 3 mCDW (risk reduction=33.0%, p=0.004) and 6 mCDW (risk reduction=31.0%, p-0.017) compared with teriflunomide. Treatment with ofatumumab demonstrated a numerically greater reduction of confirmed disability worsening (for both 3 mCDW and 6 mCDW) compared to treatment with teriflunomide in all subgroups analyzed (Table 3 and Table 4).

TABLE 3
Time to 3mCDW, by subgroup
(OMB 20 mg vs. TER 14 mg) (FAS)

| | | | KM | |
|---|---|---|---|---|
| | Event rate | | estimate at | |
| | n/N (%) | | year 2 | P-value |
| Asian | | | | |
| OMB | 3/36 | (8.3) | 9.6 | 0.776 |
| Rest of World | | | | |
| OMB | 85/910 | (9.3) | 10.9 | 0.004* |

3mCDW is defined as an increase from baseline in EDSS sustained for at least 3 months. In the time to event analysis, time is calculated as (the date of EDSS assessment at the onset of the event − date of the first administrated of study drug + 1) for patients with the events; (the date of last EDSS assessment during the treatment epoch − date of first administration of study drug + 1) for censored patients.
n: Total number of events included in the analysis.
N: Total number of patients included in the analysis.
*Indicates statistical significance (2-sided) at the 0.05 level.

TABLE 4
Time to 6mCDW, by subgroup
(OMB 20 mg vs. TER 14 mg) (FAS)

| | | | KM | |
|---|---|---|---|---|
| | Event | | estimate at | |
| | rate n/N (%) | | year 2 | P-value |
| Asian | | | | |
| OMB | 3/36 | (8.3) | 9.6 | 0.774 |
| Rest of World | | | | |
| OMB | 68/910 | (7.5) | 8.1 | 0.017* |

6mCDW is defined as an increase from baseline in EDSS sustained for at least 6 months. In the time to event analysis, time is calculated as (the date of EDSS assessment at the onset of the event − date of the first administration of study drug + 1) for patients with the events; (the date of last EDSS assessment during the treatment epoch − date of frist administration of study drug + 1) for censored patients.
n: Total number of events included in the analysis.
N: Total number of patients included in the analysis.
*Indicates statistical significance (2-sided) at the 0.05 level.

### c) Gd-Enhancing T1 Lesions

The number of Gd-enhancing T1 lesions per scan was a key secondary efficacy endpoint in the pivotal Phase III studies. In the overall population, treatment with ofatumumab significantly reduced the mean number of Gd-enhancing T1 lesions per scan compared with teriflunomide by 95.8% (p<0.001) (Table 5). In the Asian subgroups by race and region, treatment with ofatumumab demonstrated a greater reduction in the number of Gd-enhancing T1 lesions per scan compared with teriflunomide. These results were consistent with the results in the ROW subgroups and the overall population (Table 5).

TABLE 5
Gd-enhancing T1 lesions per scan, by subgroup

| | Number of Gd- | | Adjustment mean | |
|---|---|---|---|---|
| | enhancing lesions/ | | number of Gd- | Rate |
| | number of scans | | enhancing lesions | reduction (%)/ |
| | (lesion rate) | | per scan (95% Cl) | P-value |
| Asian | | | | |
| OMB | 1/54 | (0.019) | 0.02 (<0.01, 0.17) | 93.8/0.022* |
| Rest of World | | | | |
| OMB | 51/1666 | (0.031) | 0.03 (0.02, 0.04) | 95.9/<0.001* |

Gd-enhancing T1 lesion counts from scans collected within 30 days after termination of steroid therapy are excluded from the analysis.
*Indicates statistical significance (2-sided) at the 0.05 level.

### d) New or Enlarging T2 Lesions

The annualized rate of new or enlarging T2 lesions was a key secondary efficacy endpoint in the pivotal Phase III studies. In the overall population, treatment with ofatumumab significantly reduced the rate of new or enlarging T2 lesions compared with teriflunomide by 82.9% (p<0.001) (Table 6). In the Asian subgroups by race and region, treatment with ofatumumab demonstrated a greater reduction in the annualized rate of new or enlarging T2 lesions compared with teriflunomide. These results were generally consistent with the results in the rest of world subgroup and the overall population (Table 6).

TABLE 6
Annualized rate of new or enlarging T2 lesions, by subgroup

| | | | Adjusted | |
|---|---|---|---|---|
| | Number of new or | | annualized mean | |
| | enlarging lesions/ | | rate of new or | Rate |
| | patient years | | enlarging T2 | reduction |
| | (lesion years) | | lesions (95% Cl) | (%)/P-value |
| Asian | | | | |
| OMB | 44/44 | (1.00) | 1.04 (0.55, 1.96) | 59.0/0.041* |
| Rest of World | | | | |
| OMB g | 1185/1404 | (0.84 | 0.87 (0.77, 0.98) | 83.3/<0.001* |

*Indicates statistical significance (2-sided) at the 0.05 level.

### e) Adverse Events (AEs)

For the safety analyses of this report, non-serious treatment emergent adverse events (TEAEs) up to and including safety cut-off (i.e. 100 days after last study drug administration) and serious TEAEs regardless of the safety cutoff were considered (if reported prior to the data cut-off of Studies I and II). Common terminology criteria for adverse events (CTCAE) grading was used to record the severity of each AE. The latest version of CTCAE available at the time of TEAE reporting was used. The AE profile (including system organ classes (SOCs) and those patients with at least one AE) of the subgroups by race (Asian: ofatumumab 66.7% vs. teriflunomide 82.9%; ROW: ofatumumab 84.4% vs. teriflunomide 84.2%) was comparable with that of the overall population of patients in (ofatumumab 83.6% vs. teriflunomide 84.2%). The three most frequently reported AEs by SOC were ‘General disorders and administration site conditions’ ‘Infections and infestations’ and ‘Injury, poisoning and procedural complications’ in the Asian and ROW subgroups by race (Table 7). Analysis of AEs by subgroups defined by race did not suggest any meaningful difference in the types or incidences of AEs by primary SOC and by Preferred term (PT) across the treatment groups. In the Asian subgroup, 1 patient experienced SAEs of urinary tract infections, urosepsis and testicular infarction in the ofatumumab group and 1 patient reported an SAE of nephrolithiasis in the teriflunomide group (Table 8). Due to the small number of Asian patients by race and region, results should be interpreted with caution.

TABLE 7
Treatment emergent adverse events, regardless of study treatment
relationship, by primary system
organ class and subgroup - Race (Safety Set)

| | | | Rest | |
|---|---|---|---|---|
| | Asian | | of World | |
| | OMB | | OMB | |
| | N = 36 | | N = 910 | |
| Primary system organ class | n (%) | | n (%) | |
| Number of patients with at least 1 AE | 24 | (66.7) | 767 | (84.3) |
| General disorders and administration | 13 | (36.1) | 244 | (26.8) |
| site conditions | | | | |
| Infections and infestations | 8 | (22.2) | 480 | (52.7) |
| Injury, poisoning and procedural complications | 7 | (19.4) | 271 | (29.8) |
| Investigations | 6 | (16.7) | 195 | (21.4) |
| Musculoskeletal and connective tissue disorders | 5 | (13.9) | 242 | (26.6) |
| Skin and subcutaneous tissue disorders | 5 | (13.9) | 153 | (16.8) |
| Gastrointestinal disorders | 4 | (11.1) | 220 | (24.2) |
| Nervous system disorders | 4 | (11.1) | 268 | (29.5) |
| Reproductive system and breast disorders | 4 | (11.1) | 54 | (5.9) |
| Metabolism and nutrition disorders | 3 | (8.3) | 50 | (5.5) |
| Renal and urinary disorders | 3 | (8.3) | 49 | (5.4) |
| Blood and lymphatic system disorders | 3 | (8.3) | 29 | (3.2) |
| Respiratory, thoracic and mediastinal disorders | 3 | (8.3) | 103 | (11.3) |
| Ear and labyrinth disorders | 2 | (5.6) | 39 | (4.3) |
| Psychiatric disorders | 1 | (2.8) | 153 | (16.8) |
| Eye disorders | 1 | (2.8) | 54 | (5.9) |

| | | | |
|---|---|---|---|
| Vascular disorders | 0 | 55 | (6.0) |
| Hepatobiliary disorders | 0 | 17 | (1.9) |
| Immune system disorders | 0 | 16 | (1.8) |
| Cardiac disorders | 0 | 26 | (2.9) |
| Neoplasms benign, malignant and unspecified | 0 | 24 | (2.6) |
| (incl cysts and polyps) | | | |
| Endocrine disorders | 0 | 9 | (1.0) |
| Social circumstances | 0 | 3 | (0.3) |
| Congenital, familial and genetic disorders | 0 | 1 | (0.1) |

A patient with multiple AEs within a primary system organ class is counted only once in the total row.
A patient with multiple occurences of an AE under 1 treatment is counted only once in this AE category for that treatment.
System organ classes are presented in alphabetical order preferred terms are sorted within system organ class in descending frequency of AEs in the Asian Ofatumumab 20 mg treatment group.
MedDRA Version 22.0 has been used for the reporting of AEs.

TABLE 8
Serious treatment emergent adverse events, regardless of study
treatment relationship, by primary system organ
class and subgroup - Race (Safety Set)

| | | | Rest of | |
|---|---|---|---|---|
| | Asian | | the World | |
| | OMB | | OMB | |
| | N = 36 | | N = 910 | |
| Primary system organ class | n (%) | | n (%) | |
| Number of patients with at least 1 SAE | 1 | (2.8) | 85 | (9.3) |
| Blood and lymphatic system disorders | 0 | | 2 | (0.2) |
| Cardiac disorders | 0 | | 3 | (0.3) |
| Ear and labyrinth disorders | 0 | | 3 | (0.3) |
| Eye disorders | 0 | | 0 | |
| Gastrointestinal disorders | 0 | | 8 | (0.9) |
| General disorders and administration site conditions | 0 | | 3 | (0.3) |
| Hepatobiliary disorders | 0 | | 5 | (0.5) |
| Immune system disorders | 0 | | 1 | (0.1) |
| Infections and infestations | 1 | (2.8) | 23 | (2.5) |
| Injury, poisoning and procedural complications | 0 | | 13 | (1.4 |
| Investigations | 0 | | 2 | (0.2) |
| Metabolism and nutrition disorders | 0 | | 0 | |
| Musculoskeletal and connective tissue disorders | 0 | | 8 | (0.9) |
| Neoplasms benign, malignant and unspecified | 0 | | 9 | (1.0) |
| (incl cysts and polyps) | | | | |
| Nervous system disorders | 0 | | 7 | (0.8) |
| Psychiatric disorders | 0 | | 10 | (1.1) |
| Renal and urinary disorders | 0 | | 0 | |
| Reproductive system and breast disorders | 1 | (2.8) | 3 | (0.3) |

| | | | |
|---|---|---|---|
| Respiratory, thoracic and mediastinal disorders | 0 | 2 | (0.2) |
| Skin and subcutaneous tissue disorders | 0 | 1 | (0.1) |

| | | |
|---|---|---|
| Vascular disorders | 0 | 0 |

A patient with multiple AEs within a primary system organ class is counted only once in the total row.
A patient with multiple occurences of an AE under 1 treatment is counted only once in this AE category for that treatment.
System organ classes are presented in alphabetical order perferred terms are sorted within system organ class in descending frequency of AEs in the Asian Ofatumumab 20 mg treatment group.
MedDRA Version 22.0 has been used for the reporting of AEs.

### 1. Treatment Emergent Adverse Events (TEAEs)

Overall, in the Asian subgroup by race, the number of TEAEs by primary SOCs was lower in the ofatumumab group than in the teriflunomide group, except for few SOCs as listed below. There were no marked differences in AEs by primary SOC across the treatment groups in the ROW subgroup. The three most frequently reported AEs by SOC were ‘General disorders and administration site conditions’ ‘Infections and infestations’ and ‘Injury, poisoning and procedural complications’ in the Asian and ROW subgroups (Table 7).

The 3 most frequently reported AEs by PT were pyrexia (ofatumumab: 12 patients, 33.3%; teriflunomide: 5 patients, 14.3%) followed by injection-related reaction (ofatumumab: 7 patients, 19.4%; teriflunomide: 3 patients, 8.6%) followed by upper respiratory tract infection (ofatumumab: 4 patients, 11.1%; teriflunomide: 2 patients, 5.7%). The 3 most frequently reported AEs by PT were injection-related reaction (ofatumumab: 188 patients, 20.7%; teriflunomide: 140 patients, 15.5%) followed by nasopharyngitis (ofatumumab: 168 patients, 18.5%; teriflunomide: 155 patients, 17.2%), followed by alopecia (ofatumumab: 52 patients, 5.7%; teriflunomide: 138 patients, 15.3%).

### 2. Serious Adverse Events (SAEs)

The total number of patients reporting SAEs in the Asian subgroup by race was overall low and similar between the ofatumumab and teriflunomide groups in both Asian and ROW subgroup (Asian: ofatumumab: 1 patient, 2.8%; teriflunomide: 1 patient, 2.9% and ROW: ofatumumab: 85 patients, 9.3%; teriflunomide: 73 patients, 8.1%) (Table 8). In the Asian subgroup by race, in the ofatumumab group, 1 patient experienced 3 SAEs of urinary tract infections, urosepsis and testicular infarction. In the teriflunomide group, 1 patient reported SAE of nephrolithiasis.

In ROW subgroup, the most frequently reported SAE by SOC (incidences≥2% of patients) in both treatment groups was ‘Infections and infestations’ (ofatumumab: 23 patients, 2.5%; teriflunomide: 17 patients, 1.9%). The rates of SAEs by PT were generally similar across the treatment groups. None of the SAEs was reported with incidences≥1% per PT and treatment group. Appendicitis was reported in 8 patients (0.9%) in the ofatumumab group compared with 2 patients (0.2%) in the teriflunomide group. There were no clinically meaningful differences in the SAEs by PT between ofatumumab and teriflunomide groups. Except for appendicitis, only small numerical differences (<0.5%) were observed between the ofatumumab and teriflunomide groups.

### 3. AEs of Special Interest (AESI)

AEs of special interest were summarized by risk name, PT and treatment. Furthermore, the incidence of any AE that fulfilled the search terms as defined in eCRS were summarized by risk name, level, and maximum CTCAE grade. In this section, AESIs are first described for subgroups by race and then by region. There is difference of only few patients between subgroups by race and by region and therefore no meaningful difference was observed in number of AESIs in subgroups by race and by region. The definitions of AEs of special interest correspond to the identified or potential risks for ofatumumab and its class of drug as well as those for the comparator (teriflunomide). AESIs, based on the safety profiles of ofatumumab, comparator, and other anti-CD20 mABs are:
Injection-related reactions including injection-systemic reaction and injection-site
reactions
Infections, including opportunistic infections, HBV infection reactivation and PML
Malignant and premalignant disorders
Hepatic Safety
Neutropenia

### Standard Assessments of Risks

Suicidal ideation and behavior
Drug abuse and dependence
Pregnancy
Renal safety

No meaningful differences in the types or incidences of above-mentioned AEs of special interest between Asian and ROW subgroup in patients with RMS was observed. The AESI category of ‘infection’ is most frequently reported in both ofatumumab and teriflunomide groups for both Asian and ROW subgroups (Table 9).

TABLE 9
Treatment emergent adverse events, by risk name and preferred
term and subgroup - Race (Safety set)

| | Asian | | Rest of World | |
|---|---|---|---|---|
| | OMB 20 mg | | OMB 20 mg | |
| | N = 36 | | N = 910 | |
| Number of patients with | AE | | AE | |
| a least 1 AE-n (%) [E] | | | | |
| eCRS flag: SPPFL | | | | |
| Safety topic of interest: | | | | |
| Injection systemic reaction | 7 | (19.4) | 184 | (20.2) |
| (any reaction) | | | | |
| Injection-site reaction | 1 | (2.8) | 101 | (11.1) |
| (any reaction) | | | | |
| Infections | 8 | (22.2) [20] | 480 | (52.7) [1172] |
| Upper Respiratory Tract Infections | 7 | (19.4) [11] | 366 | (40.2) [643] |
| Lower Respiratory tract Infections | 0 | | 30 | (3.3) [31] |
| Urinary tract infection | 2 | (5.6) [4] | 2 | (5.7) [4] |
| Opportunistic infections | 0 | | 1 | (0.1) [1] |
| HBV Infection and Reactivation | 0 | | 0 | |
| Herpes viral infections | 0 | | 46 | (5.1) [61] |
| Varicella-Zoster virus infections | 0 | | 18 | (2.0) [19] |
| Malignant and premalignant | | | | |
| disorders | | | | |
| Risk of malignancy | 0 | | 5 | (0.5) [5] |
| Premalignant disorders | 0 | | 7 | (0.8) [9] |
| Neoplasms | 0 | | 24 | (2.6) [28] |
| Hepatic Safety | 2 | (5.6) [4] | 42 | (4.6) [60] |

| Neutropenia | 0 | 9 | (1.0) [13] |
|---|---|---|---|
| Suicidal Ideation and Behavior | 0 | 8 | (0.9) [9] |
| Drug abuse and dependence | 0 | 1 | (0.1) [1] |
| Renal Safety | 0 | 0 | |
| Pregnancy | 0 | 1 | (0.1) [1] |

N is the number of patients with the specified injections for injection-related; % = n/N.
Only systemic reactions/symptoms within 24 hours after injections were included (i.e., time to onset of reaction ≤24 hours).
AEs with a start date on or after the date of first study treatment are included.
E = number of events. E is mentioned for all risks other than injection-related reactions.
A patient with multiple AEs/SAEs is counted only once in the ‘at least 1 AE’/‘at least 1 SAE’ row.
MedDRA Version 22.1 has been used for the reporting of AEs.

### f) Region

The patterns of AESIs in the different subgroups of extrinsic factors were broadly consistent with that observed for the overall population. There was no pattern of treatment differences between the sub-groups and the pattern was generally comparable to that observed in the overall population (Table 10).

TABLE 10
Summary of adverse experiences by subgroup-Region (Safety Set)

| | Asian | | Rest of World | | Overall | |
|---|---|---|---|---|---|---|
| | OMB | | OMB | | OMB | |
| | 20 mg | | 20 mg | | 20 mg | |
| | N = 32 | | N = 914 | | N = 946 | |
| | n (%) | | n (%) | | n (%) | |
| Number of patients who experienced | 20 | (62.5) | 771 | (84.4) | 791 | (83.6) |
| at least 1 AE | | | | | | |
| Number of patients who experienced | 1 | (3.1) | 85 | (9.3) | 86 | (9.1) |
| at least 1 SAE | | | | | | |
| AESI | | | | | | |
| Injection-related reactions | | | | | | |
| Injection systemic reaction | | | | | | |
| Any injection | 5 | (15.6) | 186 | (20.4) | 191 | (20.2) |
| Injection 1 | 4 | (12.5) | 132 | (14.4) | 136 | (14.4) |
| Injection-site reaction | | | | | | |
| Any injection | 0 | | 102 | (11.2) | 102 | (10.8) |
| Injection | 0 | | 26 | (2.8) | 26 | (2.7) |
| Infections | 6 | (18.8) | 482 | (52.7) | 488 | (51.6) |
| Neoplasm | 0 | | 24 | (2.6) | 24 | (2.5) |
| Malignancies | 0 | | 7 | (0.8) | 5 | (0.5) |
| Pre-malignant disorders | 0 | | 5 | (0.5) | 7 | (0.7) |
| Hepatic safety | 2 | (6.3) | 42 | (4.6) | 44 | (4.7) |

| | | | | | |
|---|---|---|---|---|---|
| Neutropenia | 0 | 9 | (1.0) | 9 | (1.0) |
| Standard assessment of risks | | | | | |
| Suicidal ideation and behavior | 0 | 8 | (0.9) | 8 | (0.8) |
| Pregnancy | 0 | 1 | (0.1) | 1 | (0.1) |

| | | | | |
|---|---|---|---|---|
| Drug abuse and dependence | 0 | 1 | (0.1) | 0 |

| | | | |
|---|---|---|---|
| Renal safety | 0 | 0 | 0 |

Events are identified and SOC is determined using the CRS
Only primary SOC was reported for the safety topic of interest: ‘Infections’ and ‘Neoplasms’

### Example 2: Long Term Treatment and Effect on Ig Levels

Patients were subjected to a long term treatment. Patients received ofatumumab 20 mg sc injections every 4 weeks (after an initial loading regimen of 20 mg sc doses on weeks 0, 1 and 2).

### Duration of Exposure was as Follows:

| | Long-term | | Newly-switched | | Overall | |
|---|---|---|---|---|---|---|
| Duration of exposure | N = 1026 | | N = 677 | | N = 1703 | |
| Any exposure | 1026 | (100 ) | 677 | (100) | 1703 | (100) |
| <48 weeks (1 year) | 13 | ( 1.3) | 63 | ( 9.3) | 76 | ( 4.5) |
| 48 weeks to 96 weeks | 260 | ( 25.3) | 614 | ( 90.7) | 874 | ( 51.3) |

| (1 to 2 years) | | | | | |
|---|---|---|---|---|---|
| 96 weeks to 144 | 235 | ( 22.9) | 0 | 235 | ( 13.8) |
| weeks (2 to 3 years) | | | | | |
| 144 weeks to 192 | 466 | ( 45.4) | 0 | 466 | ( 27.4) |
| weeks (3 to 4 years) | | | | | |
| > 192 weeks (4 years) | 52 | ( 5.1) | 0 | 52 | ( 3.1) |

| | | | |
|---|---|---|---|
| Exposure in months | | | |
| N | 1026 | 677 | 1703 |
| Mean | 30.9 | 15.3 | 24.7 |
| SD | 9.26 | 3.61 | 10.72 |
| Minimum | 3.7 | 1.0 | 1.0 |
| Q1 | 21.8 | 14.1 | 16.9 |
| Median | 33.2 | 16.6 | 20.4 |
| Q3 | 38.1 | 17.6 | 35.0 |
| Maximum | 50.5 | 20.2 | 50.5 |
| Patient-years | 2637.7 | 863.0 | 3500.7 |

Demographics were as Follows:

| | Long-term | Newly-switched | Overall |
|---|---|---|---|
| Characteristic | N = 1026 | N = 676 | N = 1702 |
| Age group - n (%) | | | |
| 18 to 30 years | 245 ( 23.9) | 116 ( 17.2) | 361 ( 21.2) |
| 31 to 40 years | 378 ( 36.8) | 239 ( 35.4) | 617 ( 36.3) |
| 41 to 55 years | 400 ( 39.0) | 287 ( 42.5) | 687 ( 40.4) |
| >55 years | 3 ( 0.3) | 34 ( 5.0) | 37 ( 2.2) |

### Results

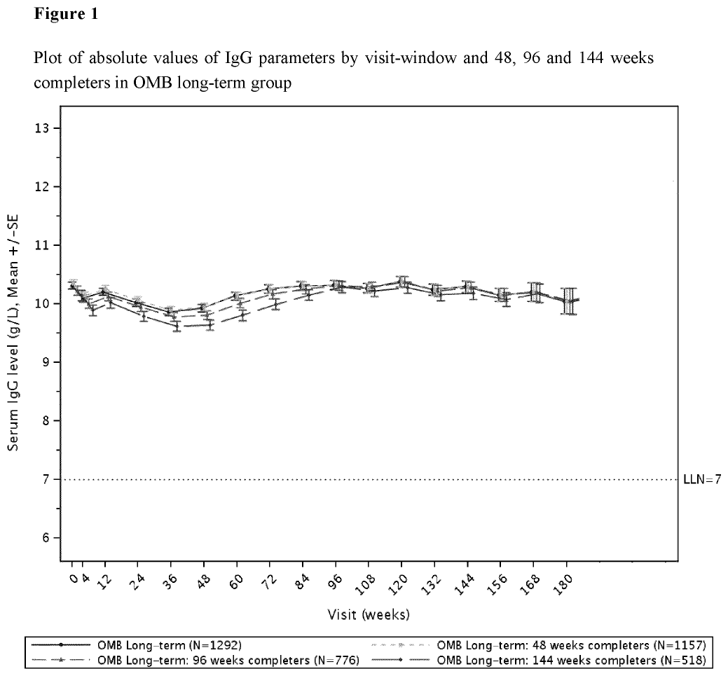

The results on IgG and IgM levels were shown in FIGS. 1-4.

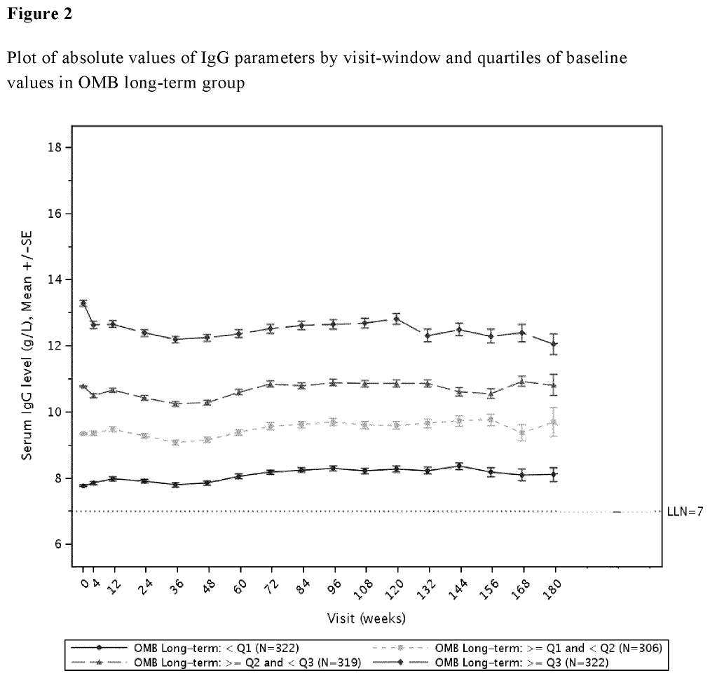

FIG. 1 shows that IgG levels were stable over the long-term follow-up of >4 years. This is of particular importance for the lower quartile, see FIG. 2.

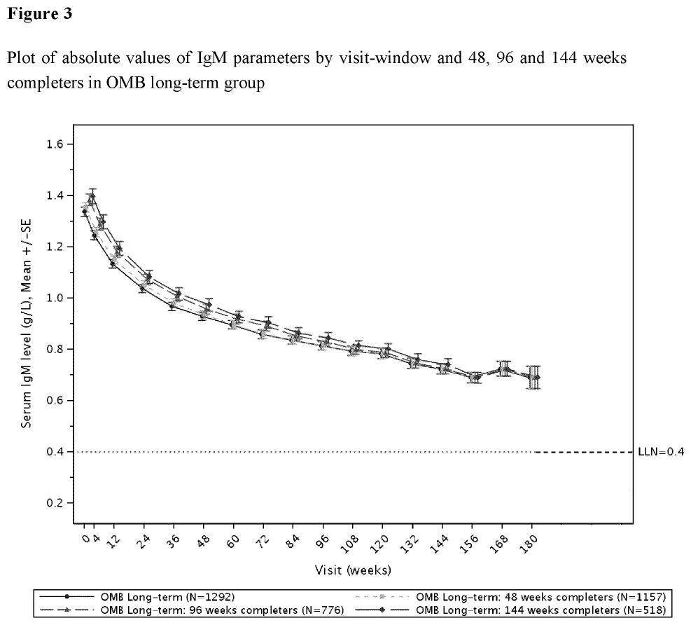

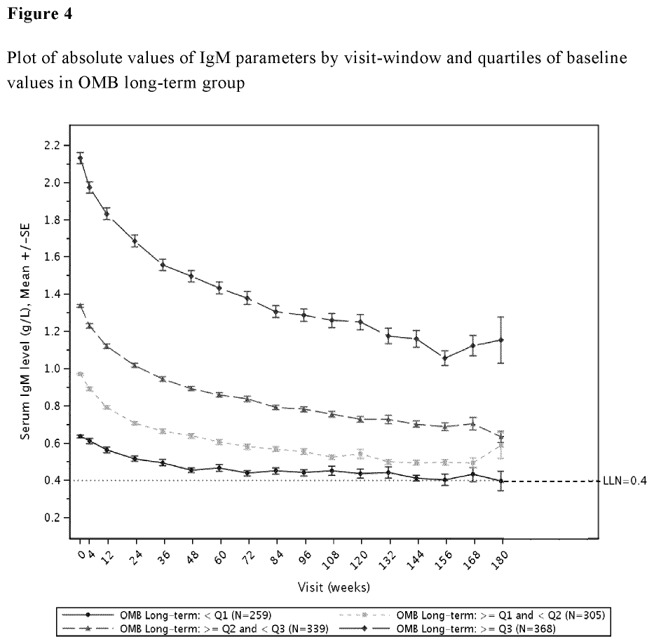

A reduction in IgM levels were seen with longer duration; however, levels remained above the lower limit of normal, see FIGS. 3 and 4.

### Example 3

Characteristics and Outcome of COVID-19 in Patients with Relapsing Multiple Sclerosis Receiving Ofatumumab
Objective: To report clinical characteristics of COVID-19 infection in people with MS (pwMS) receiving ofatumumab 20 mg subcutaneously every 4 weeks

### Methods:

Confirmed or suspected cases of COVID-19 infection in patients receiving ofatumumab in the open-label extension study ALITHIOS were reviewed (data cutoff: Dec. 21, 2020)
COVID-19 cases were classified as confirmed if SARS-CoV2 positive test results were available, or patient was reported to be diagnosed with COVID-19
Suspected cases of COVID-19 were classified as suspected if without a positive SARS-CoV2 test or definitive diagnosis
   The Following COVID-19 Case Characteristics were Assessed:
Patient demographics
COVID-19 seriousness category*
Ofatumumab treatment duration and action taken with ofatumumab (treatment interruption)
Interventions and COVID-19 outcomes
*Serious criteria is based on the regulatory reporting definition established by ICH, for the purposes of regulatory reporting obligations, and consists of fatal, life-threatening, hospitalization and medically significant
Patient characteristics: Overall population
Patient demographics and drug exposure are shown in the table below: 45% (466/1026) of patients in the long-term group have drug exposure of 3-4 years Over 90% (614/677) of patients in the newly switched group have ofatumumab exposure of 1-2 years

| | Ofatumumab - | Ofatumumab - | |
|---|---|---|---|
| | long term group | newly switched | Overall |
| Characteristics | N = 1026 | N = 677 | N = 1703* |
| Age, mean, years | 38 | 40 | 39 |
| Age group, % of | | | |
| patients | | | |
| 18-30 | 24 | 17 | 21 |
| 31-40 | 37 | 35 | 36 |
| 41-55 | 39 | 43 | 41 |
| >55 | 0.3 | 5 | 2 |
| Sex, n (%) | | | |
| Male | 296 (29) | 220 (33) | 516 (30) |
| Female | 730 (71) | 456 (68) | 1186 (70) |
| Exposure, months | | | |
| Mean | 30.9 | 15.3 | 24.7 |
| Range | 3.7-50.5 | 1.0-20.2 | 1.0-50.5 |
| Patient-years | 2637.7 | 863.0 | 3500.7 |

### Results: COVID-19 Cases Overview

As of 21 Dec. 2020, 35 out of 1703 patients had a confirmed COVID-19 infection and/or COVID-19 pneumonia in the ongoing open-label, extension ALITHIOS clinical trial for ofatumumab (Table)

All the non-serious cases were reported as completely recovered

For 21 cases no change in ofatumumab treatment required and for 5 cases treatment was temporarily interrupted due to confirmed COVID-19 infection

Of the 6 serious cases, 5 had complete recovery and in one patient the COVID-19 outcome was fatal (details as below).

The 48 year-old patient, with no associated risk factors* (*relevant comorbidities, such as chronic lung condition, diabetes, hypertension or malignancy), reported symptoms of COVID-19 (pneumonia, fever, weakness, cough and dyspnoea) after approximately 3 years and 7 months of ofatumumab treatment. The patient was hospitalized and received steroids, antivirals, antibiotics and COVID-19 convalescent plasma. The COVID-19 outcome was reported as not related to ofatumumab treatment.

| Confirmed cases | N = 35 |
|---|---|
| Non-serious (n = 29) | |
| Age, mean (range), years | 35.6 |
| | (range 28-50) |
| Sex | |
| Female | 17 |
| Male | 12 |
| COVID-19 outcome | |
| Recovered | 29 |
| Recovering | 0 |
| Ongoing | 0 |
| Serious (n = 6) | |
| Age, mean (range), years | 46 |
| | (range 38 to 57) |
| Sex | |
| Female | 3 |
| Male | 3 |
| COVID-19 outcome | |
| Recovered | 5 |
| Fatal | 1 |

### Results: COVID-19 Serious Cases

Characteristics of the six serious cases of COVID-19 in patients receiving ofatumumab is listed below:

| | | | | OMB | | | | | | |
|---|---|---|---|---|---|---|---|---|---|---|
| | | | | treatment | | | | COVID-19 | | |
| | | | Duration | during | | Relevant | | symptoms | | |
| Patient | Age | | of OMB | COVID-19 | COVID-19 | medical | COVID-19 | duration | | |
| ID | (years) | Sex | treatment | infection | symptoms | conditions | treatment | (days) | | Outcome* |
| 5409018 | 39 | F | ~3 y 1 m | Interrupted | Bilateral | H/O | Hydroxychloroquine, | ~50 | d | Complete |
| | | | | | pneumonia | respiratory | Antivirals, | | | recovery |
| | | | | | | infection | Antibiotics | | | |
| 5608014 | 46 | M | ~2 y 11 m | Interrupted | Fever, sore | Arterial | Dexmethasone, | 18 | d | Complete |
| | | | | | throat, | hypertension | Antivirals, | | | recovery |
| | | | | | malaise, | | Antibiotics | | | |
| | | | | | pneumonia | | | | | |
| 5800043 | 57 | M | ~1 y 4 m | Continued | Fever, | Not reported | Not reported | 11 | d | Complete |
| | | | | | weakness, | | | | | recovery |
| | | | | | dyspnea, | | | | | |
| | | | | | pneumonia | | | | | |
| 5806001 | 38 | M | ~3 y 6 m | Continued | Fever, cough, | Pneumocystis | Steroids, | 10 | d | Complete |
| | | | | | weakness, | jirovecii | antibiotics | | | recovery |
| | | | | | bilateral | pneumonia | | | | |
| | | | | | interstitial | | | | | |
| | | | | | pneumonia | | | | | |
| 5801015 | 53 | F | ~3 y 4 m | Interrupted | Fever, chest | Upper | Remdesivir | 15 | d | Complete |
| | | | | | pain, nausea, | respiratory | | | | recovery |
| | | | | | pneumonia, | tract infection | | | | |
| | | | | | dyspnea | | | | | |
| 5800004 | 48 | F | ~3 y 9 m | Drug | Pneumonia, | H/O | Remdesivir, | ~30 | d | Fatal |
| | | | | withdrawn | fever, cough, | hepatopathy, | COVID-19 | | | |
| | | | | | weakness, | allergy, upper | convalescent | | | |
| | | | | | dyspnea | respiratory | plasma, | | | |
| | | | | | | tract infection | steroids, | | | |
| | | | | | | | antibiotics | | | |

Ofatumumab treatment was continued in two and temporarily interrupted in three patients with COVID 19
Except one case, where information was not reported, all cases had pre-existing comorbidities identified as risk factors for severe COVID-19 outcome in general population (i.e., respiratory disease and hypertension)
Ofatumumab treatment was reported as not related with the COVID-19 course or outcome in all six cases
COVID-19, coronavirus disease 2019;
d, days;
H/O, history of;
m, months;
OMB, ofatumumab;
f, female;
m, male;
y, years;
*Last available information Outcomes available at the time of the most recent follow up

### Conclusions

Of the total 1703 patients in the ongoing ALITHIOS study, 35 confirmed cases of COVID-19 infection and/or COVID-19 pneumonia have been reported. Complete recovery in 34 cases; one case had fatal outcome. None of the cases were suspected because of ofatumumab treatment.

Based on the review of reported cases, the clinical presentations and outcomes of COVID-19 cases in pwMS on ofatumumab therapy was similar to other reports on MS population (Richadson S, et al. JAMA. 2020; 323:2052-2059; Sormani M P, et al. Lancet Neurol. 2020 June; 19(6):481-482; Montero-Escribano P, et al. Mult Scler Relat Disord. 2020; 42:102185; Safavi F, et al. Mult Scler Relat Disord. 2020; 43:102195; Barzegar M, et al. Mult Scler Relat Disord. 2020; 45:102276) and in the general population (Bchetnia M, et al. J Infect Public Health. 2020; 13(11):1601-1610).

1. Ofatumumab for use in the treatment of multiple sclerosis, wherein patients are treated who are of Asian race.

2. Ofatumumab for use in the treatment of multiple sclerosis according to claim 1, wherein patients are treated who
harbor the CYP 2C9*2 allele of cytochrome P450 (CYP) at a frequency of less than 10%, preferably less than 5%, more preferably less than 4.5%, even more preferably less than 4.4%, most preferably less than 4%, and/or
have epidermal growth factor receptor (EGFR) mutations and/or
have the VKORC1 low warfarin dose haplotype and/or
possess the promyelocytic leukemia protein (PML) gene breakpoint cluster region-1 subtype (bcr1) of chromosomal translocation t(15:17).

3. Ofatumumab for use in the treatment of multiple sclerosis according to claim 1 or 2, wherein the patients
have neuromyelitis optica (NMO) and/or
have NMO spectrum disorders and/or
have a history of sudden death (Brugada syndrome) in their family and/or
have cardiac sodium channelopathy from SCN5A loss-of-function mutation with arrthymogenic susceptibility,
have thyrotoxic periodic paralysis and/or
are obese (preferably central obesity characterized by a waist circumference >80 cm in women and >90 cm in men, more preferably combined with a “thin outside fat inside (TOFI)” characteristic phenotype of obesity) and/or
have type 2 diabetes mellitus and/or
have diabetic nephropathy and/or
have aggressive triple negative or basal breast cancer (ER, PR, Her2/neu negative) and/or
have a history of lacunar strokes and/or
have a history of intracerebral hemorrhage and/or
have systemic lupus erythematosus (SLE) and/or
suffer from obstructive sleep apnea (OSA) and/or
have nasopharyngeal cancer (NPC), preferably differentiated non-keratinizing carcinoma (WHO Type 2 histology) and/or
have insulin resistance.

4. Ofatumumab for use in the treatment of multiple sclerosis, wherein the treatment is ethnically insensitive.

5. Ofatumumab for use in the treatment according to any of the preceding claims, wherein the adverse events occurring in patients of Asian race are consistent with the overall safety profile of ofatumumab.

6. Ofatumumab for use in the treatment according to any of the preceding claims, wherein the adverse events are selected from injection-related reactions, infections, malignant and premalignant disorder, hepatic damage or dysfunction and neutropenia.

7. Ofatumumab for use in the treatment according to claim 6, wherein the infections are selected from respiratory tract infections, urinary tract infections, Herpes viral infections, Varicella-Zoster infections, opportunistic infections, pulmonary tuberculosis, HBV infection reactivation and progressive multifocal leukoencephalopathy (PML).

8. Ofatumumab for use in the treatment according to claim 6 or 7, wherein the infections are serious infections, preferably selected from opportunistic infections, HBV infection reactivation, Pneumocystis jirovecii pneumonia (PCP) and PML.

9. Ofatumumab for use according to any one of the preceding claims, wherein the patients are switched from an earlier disease modifying treatment (DMT) to ofatumumab, wherein the switch is preferably made when there is a change in
the earlier DMT's Cₘₐₓ, and/or
the earlier DMT's AUC, and/or
B cell and/or T cell inhibition or depletion, and/or
serum IgG level, and/or
adverse events.

10. Ofatumumab for use in the treatment according to any one of the preceding claims, wherein serum IgG levels are maintained during the treatment within a range from 500 to 1800 mg/dl.

11. Ofatumumab for use in the treatment according to any one of the preceding claims, wherein the treatment is a long-term treatment.

12. Ofatumumab for use according to any one of the preceding claims, wherein ofatumumab is administered at a dose of 10 to 30 mg every 4 weeks, preferably 20 mg every 4 weeks.

13. Ofatumumab for use according to any one of the preceding claims, wherein ofatumumab is administered subcutaneously.

14. Ofatumumab for use according to any one of the preceding claims, wherein ofatumumab is administered with a loading dose.

15. Ofatumumab for use according to claim 14, wherein 20 mg ofatumumab at weeks 0, 1 and 2 is administered as a loading dose.

16. Ofatumumab for use according to any one of the preceding claims, wherein multiple sclerosis is selected from relapsing multiple sclerosis, in particular clinically isolated syndrome (CIS), relapsing remitting multiple sclerosis (RRMS) and secondary progressive multiple sclerosis (SPMS).

17. Ofatumumab for use according to any one of the preceding claims, wherein multiple sclerosis is selected from primary progressive multiple sclerosis (PPMS) or progressive relapsing multiple sclerosis (PRMS).

18. Ofatumumab for use according to any one of the preceding claims, wherein a premedication is administered to the patient before the first dose of ofatumumab is administered.

19. Ofatumumab for use according to claim 18, wherein the premedication comprises acetaminophen, antihistamines and/or steroids.

20. Ofatumumab for use according to claim 18 or 19, wherein the premedication is administered 30 to 60 minutes prior to an ofatumumab injection.

21. Ofatumumab for use according to any one of claims 1 to 17, wherein no premedication is administered prior to the first dose of ofatumumab.

22. Ofatumumab for use according to any one of the preceding claims, wherein a patient acutely or previously infected by COVID-19 is treated.

23. Ofatumumab for use according to any one of the preceding claims, wherein the treatment is continued during COVID-19 infection.

24. Ofatumumab for use according to any one of claims 1 to 22, wherein the treatment is interrupted during COVID-19 infection und continued after overcoming the infection.

25. Ofatumumab for use according to any one the preceding claims, wherein patients are treated who are of Chinese, Japanese or Korean race.
